(19) United States
(12) Patent Application Publication
(10) Pub. No.: US 2026/0207965 A1
(43) Pub. Date: Jul. 23, 2026

(54) SYSTEMS AND METHODS FOR INDEX-MATCHED OPTICAL COUPLING FOR ENHANCED PHOTOBIOMODULATION LIGHT DELIVERY TO SKIN

(71) Applicants: Joseph MERCOLA, Ormond Beach, FL (US); Christopher William JOHNSON, Fort Lauderdale, FL (US)

(72) Inventors: Joseph MERCOLA, Ormond Beach, FL (US); Christopher William JOHNSON, Fort Lauderdale, FL (US)

(21) Appl. No.: 19/455,688

(22) Filed: Jan. 21, 2026

Related U.S. Application Data
(60) Provisional application No. 63/748,168, filed on Jan. 22, 2025.

(51) Int. Cl. A61N 5/06; A61N 5/067; A61N 5/00

(52) U.S. Cl. CPC A61N 5/0616; A61N 5/067; A61N 2005/005; A61N 2005/0626; A61N 2005/0632; A61N 2005/0652; A61N 2005/0664

(57) ABSTRACT

Systems, devices, and methods for delivering light to skin with enhanced optical coupling efficiency for photobiomodulation are described. A light emitter is configured to emit light. A conformable index-matching medium, which may comprise silicone and hydrogel with refractive indices matched to skin tissue, is coupled to the light emitter on one side and contacts the skin on another side. Air is removable from between the medium and skin to create an index-matched optical pathway that reduces Fresnel reflection losses. The medium receives light from the light emitter and delivers the light through the index-matched interface to enhance penetration into the skin for photobiomodulation. Structural support elements, cooling systems, air evacuation mechanisms, and controllers may be integrated for optimal device performance.

### CROSS REFERENCE TO RELATED APPLICATIONS

This application claims priority to U.S. Provisional Patent Application No. 63/748,168, filed January 22, 2025, which is incorporated herein by reference in its entirety.

### BACKGROUND

The present invention relates generally to the fields of optical devices and wellness devices, and more particularly to innovations in delivering low-level light to skin through index-matched optical coupling interfaces. Certain aspects of the present disclosure relate to using conformable index-matching materials to optimize delivery of light to skin for photobiomodulation.

Photobiomodulation (PBM) is a non-invasive approach that employs low-level or low-intensity light to support cellular function. PBM has been studied in various contexts including skin wellness, cellular function support, and light-based skincare applications. The light used in PBM, typically in the red and near-infrared spectrum, penetrates the skin and underlying tissues. PBM may support mitochondrial function and cellular metabolism. The light is believed to interact with intracellular chromophores, which may enhance cellular activity. Existing devices for PBM range from large light panels to handheld units, with varying levels of precision and effectiveness based on parameters such as wavelength, intensity, and duration.

Currently available PBM devices often suffer from several limitations. One common disadvantage is the lack of uniform light distribution over the target area, which can lead to inconsistent outcomes. Many devices rely on static application methods or require manual repositioning, increasing the risk of operator error and reducing efficacy. Additionally, some PBM devices deliver light at intensities that can be either insufficient for benefit or excessively high, potentially causing discomfort or excessive heat generation. Many models are bulky, expensive, and difficult to operate without professional training, which restricts their adoption for home use or in low-resource settings.

A fundamental limitation of existing PBM devices is the air-skin interface present when light is delivered through an air gap between the device and the skin surface. When light travels from a device through air before entering skin tissue, significant optical losses occur due to refractive index mismatches at the boundary interfaces. The refractive index of air is approximately 1.0, while human skin tissue has a refractive index of approximately 1.4 to 1.55 depending on the tissue layer. This substantial mismatch causes reflection, refraction, and scattering of incident light at the air-skin boundary, reducing the amount of light that penetrates into target tissues. These reflections are commonly known as Fresnel reflections, which occur at any interface between materials with different refractive indices. Furthermore, the microscopic surface roughness of skin creates additional scattering effects when light must traverse an air gap before contact with the irregular skin surface.

Many handheld PBM systems, while more convenient than larger panels, often lack optical coupling mechanisms that eliminate the air-skin interface needed for efficient deep-tissue light penetration. While some existing devices use index-matching fluids or gels applied to the skin surface, these liquid approaches present practical challenges including mess, evaporation, inconsistent application thickness, and the need for repeated reapplication during sessions. These shortcomings underscore the need for innovative solutions that enhance the optical coupling efficiency of PBM devices, thereby broadening their applications and accessibility while improving outcomes.

### SUMMARY OF THE INVENTION

In one aspect, a system for delivering light to skin is disclosed that includes a light emitter configured to emit light, and a medium coupled to the light emitter on one side and in contact with the skin on another side such that air is removable from between the medium and the skin. The medium may be configured to receive the light from the light emitter and deliver the light to the skin for photobiomodulation.

The medium comprises silicone and hydrogel. A ratio of the silicone to the hydrogel in the medium is between approximately one half (1/2) and approximately three quarters (3/4) by volume. The medium may include a thin layer of silicone configured to be in direct contact with the skin.

The hydrogel is in direct contact with the skin. The light emitter is an array of light-emitting diodes. The array may be arranged in a hexagonal shape. The array may be arranged in a circular shape.

The light emitter comprises at least one laser diode or at least one diffuser. The medium may include silicone baffles.

The system may include a microfluidic channels coupled to at least one of the light emitter and the medium for cooling. The system may include a cooling fan. The system may include a one-way valve to allow the air to be removed from an interface between the medium and the skin. The system may include a controller configured to operate the light emitter and wirelessly receive control signals from a user device. The user device may be a mobile device. The controller may operate the light emitter in pulsed mode or continuous mode.

In another aspect, a device for delivering light to skin is disclosed that includes a light emitter configured to emit light, and a medium coupled to the light emitter on one side and in contact with the skin on another side. Air may be removed from between the medium and the skin. The medium may be configured to receive the light from the light emitter and deliver the light to the skin for photobiomodulation.

In yet another aspect, a method for delivering light to skin for photobiomodulation is disclosed. The method includes emitting light via a light emitter and removing air between a medium and the skin. The medium may be coupled to the light emitter on one side and in contact with the skin on another side. The method may include receiving, by the medium, light from the light emitter, and delivering the light to the skin for photobiomodulation.

### BRIEF DESCRIPTION OF THE DRAWINGS

FIG. 1 is a perspective view of a system environment (100) for delivering light to skin (110) for photobiomodulation, showing the system environment (100) with skin (110), medium (120), light emitter (130), cooler (140), controller (150), and user device (160).

FIG. 2A is a cross-sectional view of a medium (230) with a thin silicone layer (210) interfacing with skin (110), showing the thin silicone layer (210), LED array (220), medium (230), hydrogel (240), and microfluidic channels (250).

FIG. 2B is a cross-sectional view of hydrogel (260) in a medium (270) interfacing directly with skin (110), showing hydrogel (260), medium (270), LED array (280), and silicone (290) forming baffles or prongs.

FIG. 3 is a flowchart depicting a process (300) for delivering light to skin for photobiomodulation, showing step (310) for emitting light via a light emitter, step (320) for removing air between a medium and skin, step (330) for receiving light by the medium from the light emitter, and step (340) for delivering light to the skin for photobiomodulation.

FIG. 4 is a detailed cross-sectional view showing a coupling interface (420) that can be implemented with the system environment of FIG. 1. The coupling interface includes a silicone-hydrogel composite structure including thin silicone layer (412), hydrogel region (414), structural baffles (416) with integrated microfluidic channels (418) and vent paths (419), evacuation ring chamber (421) with one-way valve (422), optical sensor element (430), pressure sensor element (440), and optional diffuser (435) upstream of coupling interface.

FIG. 5 is a block diagram of a system block diagram illustrating photobiomodulation system (500) showing light emitter (510), coupling interface (520), coupling establishment subsystem (525), coupling verification sensors (550, 555), controller (560), and skin interface (570), with signal paths showing the coupling verification loop, emission gating, and dose normalization feedback. The controller (560) includes a processor (560A), memory (560B), communication module (560C), power management circuit (560D), sensor interface (560E), light emitter driver (560F), cooling control module (560G), and user interface connection (560H).

FIG. 6 is a diagram illustrating replaceable cartridge system (600) showing disposable coupling cartridge (610) with silicone-hydrogel pad (612), baffles (616), evacuation network (640), alignment interface (620), and reference region (645), mating with reusable emitter head (650) including LED array (652) and head-mounted reference region (647).

FIG. 7 is a dose normalization graph (700) illustrating the relationship between coupling metric axis (710) on the horizontal axis and compensation factor axis (720) on the vertical axis, showing compensation function curve (730), ceiling (740), coupling threshold (750), abort/reposition zone (760), and normal operating zone (770).

FIG. 8 is a method flowchart depicting process (S800) showing step (S810) for positioning coupling interface, step (S820) for establishing enhanced coupling, step (S830) for measuring coupling quality via test emission, step (S835) for determining coupling metric, decision step (S840) for threshold verification with safety limit check (S842), step (S850) for enabling therapeutic emission with dose normalization, and step (S860) for maintaining and re-verifying coupling during treatment.

### DETAILED DESCRIPTION OF THE INVENTION

The following detailed description refers to the accompanying drawings. Wherever possible, the same reference numbers are used in the drawings and the following description to refer to the same or similar parts. While several illustrative embodiments are described herein, modifications, adaptations and other implementations are possible. For example, substitutions, additions or modifications may be made to the components illustrated in the drawings, and the illustrative methods described herein may be modified by substituting, reordering, removing, or adding steps to the disclosed methods. Accordingly, the following detailed description is not limited to the disclosed embodiments and examples. The proper scope is defined by the appended claims.

Embodiments of the present disclosure describe systems, devices, methods, and compositions for delivering light to skin with enhanced optical coupling efficiency. A light emitter may be configured to emit light in the visible to near-infrared spectrum suitable for photobiomodulation. A conformable index-matching medium may be coupled to the light emitter on one side and configured to contact the skin on another side, with air being removable from between the medium and the skin surface. The medium may be configured to receive the light from the light emitter and deliver the light through an index-matched optical pathway to enhance light penetration into the skin for photobiomodulation.

In various embodiments, the index-matching medium comprises a combination of silicone and hydrogel materials having refractive indices substantially matched to that of skin tissue. By matching the refractive index of the medium to that of skin, Fresnel reflection losses at the medium-skin interface may be substantially reduced compared to an air-skin interface. The medium may include one or more structural supports, such as silicone baffles or prongs, extending through the hydrogel to provide mechanical stability while maintaining optical transparency. The conformable nature of the medium allows it to adapt to the irregular surface topology of skin, ensuring intimate contact that displaces air and creates a continuous optical pathway from the light source to the tissue.

Embodiments may include air evacuation mechanisms such as one-way valves, vacuum systems, or manual pumping devices to actively remove air trapped at the interface between the medium and the skin surface. Thermal management features such as microfluidic cooling channels, cooling fans, and heat exchangers may be integrated to dissipate heat generated by the light emitter and maintain desired operating temperatures.

The light emitter may comprise various configurations including arrays of light-emitting diodes arranged in hexagonal, circular, or other geometric patterns optimized for uniform light distribution, laser diodes for coherent light delivery, or combinations thereof. Optical elements such as diffusers, collimators, and lenses may be incorporated to control beam characteristics. Controllers may operate the light emitter in continuous waves, pulsed, or modulated modes with adjustable parameters, and may communicate wirelessly with user devices such as mobile phones, for remote operation and monitoring.

Manufacturing methods for producing the index-matching medium and integrated device assemblies are disclosed, including overmolding, coating, lamination, and polymerization techniques.

As used herein, unless specifically stated otherwise, the term "or" encompasses all possible combinations, except where infeasible. For example, if it is stated that a component may include A or B, then, unless specifically stated otherwise or infeasible, the component may include A, or B, or A and B. As a second example, if it is stated that a component may include A, B, or C, then, unless specifically stated otherwise or infeasible, the component may include A, or B, or C, or A and B, or A and C, or B and C, or A and B and C.

As used herein, the term "approximately" or "about" means within plus or minus ten percent of the stated value, unless otherwise specified. For example, "approximately 100 nm" encompasses the range from 90 nm to 110 nm.

As used herein, the term "index-matching" refers to the selection of materials having refractive indices that are sufficiently similar to reduce optical reflection and refraction losses at interfaces between the materials. In some embodiments, two materials may be considered index-matched when their refractive indices differ by less than approximately 0.2 at the wavelengths of interest. In some embodiments, the refractive indices may differ by less than approximately 0.1.

As used herein, the term "coupling metric" refers to a quantified value indicative of the optical transmission efficiency at the interface between a coupling interface and skin. The coupling metric may be expressed as a coupling transmission coefficient representing estimated fractional transmission, as a correction factor derived from sensor measurements, as an index into a lookup table mapping sensor values to compensation parameters, or as any other suitable quantification. The coupling metric is derived from one or more sensor measurements including optical reflectance or backscatter intensity, boundary pressure or seal integrity, electrical impedance or capacitance, or combinations thereof.

As used herein, the term "coupling verification sensor" refers to any sensor that generates a signal usable to determine the coupling metric, including without limitation optical reflectance sensors, optical backscatter sensors, pressure sensors, leak-rate sensors, electrical impedance sensors, electrical capacitance sensors, acoustic sensors, and combinations thereof.

As used herein, the term "verified coupling state" refers to a condition in which the coupling metric has been computed from sensor measurements and determined to satisfy a predetermined threshold indicative of adequate optical coupling for therapeutic delivery.

As used herein, the term "tissue-entry dose" refers to the radiant exposure or fluence delivered at the tissue boundary—that is, the energy per unit area crossing from the coupling interface into the skin surface. Tissue-entry dose is distinguished from deeper subsurface dose, which additionally depends on tissue optical properties. In some embodiments, tissue-entry dose is estimated from emitted energy and the coupling metric, optionally calibrated using a reference region measurement, and recorded by the controller. The target tissue-entry dose may be predetermined, user-selected, clinician-selected, protocol-selected, or adaptively selected by the controller.

As used herein, the term "dose normalization" refers to the adjustment of emitter power, duty cycle, exposure duration, or combination thereof based on the measured coupling metric such that the tissue-entry dose approximates a target value despite variations in interface transmission efficiency.

As used herein, the term "therapeutic emission" refers to light emission at power levels and durations intended to produce therapeutic photobiomodulation effects. This is distinguished from "test emission" or "diagnostic emission," which refers to low-power light pulses used for coupling verification measurements. Test emission may occur while the therapeutic gate is closed (coupling not yet verified); therapeutic emission is permitted only when coupling is verified. In some embodiments, test emission has an energy (radiant exposure) less than a predetermined fraction of the prescribed therapeutic session dose, such as less than 1%, and is insufficient to materially contribute to the therapeutic protocol.

The efficacy of photobiomodulation (PBM) may be influenced by the efficient delivery of light energy to target tissues, which can be significantly affected by the interface and mediums between the light source and the skin (110). Traditional devices often encounter challenges such as light scattering, reflection, and energy loss at the air-skin interface, resulting in reduced PBM effectiveness. When light passes from air (refractive index approximately 1.0) to skin (refractive index approximately 1.4-1.55), Fresnel reflection causes a portion of the light to be reflected back rather than transmitted into the tissue. By incorporating conformable refractive index-matching materials in medium (120) between the light source and the skin (110), these Fresnel reflection losses may be substantially reduced. Index-matching materials in medium (120) minimize the refractive discontinuity between the device and biological tissue, reducing light reflection and maximizing transmission and light penetration into target tissues. The disclosed embodiments improve not only the overall efficiency of PBM but also allow for more consistent and reproducible outcomes, paving the way for more reliable and effective applications across various wellness and cosmetic domains.

In some embodiments, PBM may support mitochondrial function and cellular metabolism. in some embodiments, LED and laser light at wavelengths in the red (approximately 620-700 nm) and near-infrared (approximately 700-1400 nm) spectrum may interact with mitochondrial components and support cellular processes. Different wavelengths may engage distinct primary photoacceptors, vary in tissue penetration depth, and elicit different cellular responses depending on the target tissue type.

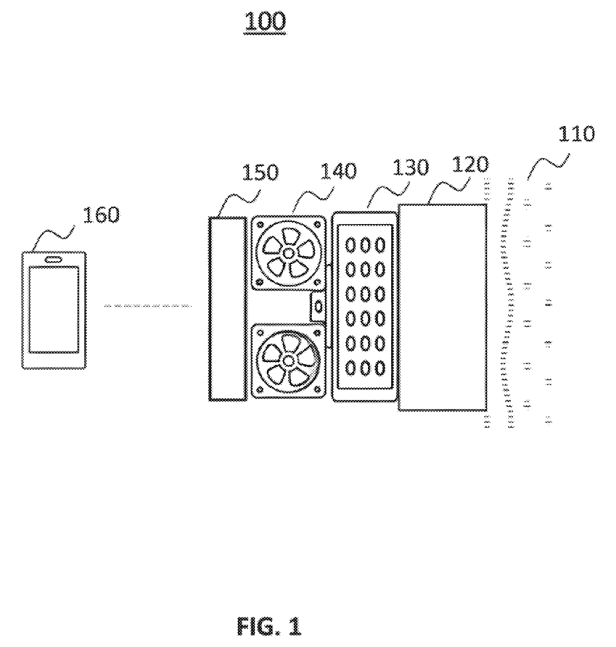

Referring now to FIG. 1, a system environment (100) for delivering light to skin (110) is illustrated. Skin (110) may be in direct contact with medium (120), with air being substantially removed from the interface to create an index-matched optical coupling. Medium (120) may include various materials which are selected for their optical properties, including refractive index, transparency, and biocompatibility, as well as structural properties including conformability, mechanical stability, and durability. Materials in medium (120) may exhibit high optical transmittance, for example, greater than approximately 80% at relevant wavelengths, to allow light from light emitter (130) to traverse medium (120) with minimal absorption losses to reach skin (110). Heat generated during emission of light or other components associated with system environment (100) may be released using cooler (140). Cooler (140), light emitter (130), or any other electrical component of the system may be controlled by controller (150). Controller (150) may also communicate with user device (160), for example wirelessly, which may be configured to operate controller (150), light emitter (130), or cooler (140).

Light emitter (130) may be configured to emit various forms of light, including but not limited to coherent light, such as that produced by lasers or laser diodes, and non-coherent light, such as that produced by light-emitting diodes (LEDs). A laser diode may be a compact semiconductor version of a laser suitable for integration into portable devices. In some embodiments, light emitter (130) includes at least one laser diode. This may include an array of laser diodes of various wavelengths. An LED may be a semiconductor device that emits non-coherent, broader spectrum light when an electrical current passes through it. In some embodiments, light emitter (130) is an array of LEDs. The array may be arranged in various patterns, including a hexagonal or circular shape or pattern, a rectangular grid, a concentric ring arrangement, or other geometric configurations optimized for uniform light distribution.

Hexagonal geometry in light emitter (130) may increase the packing factor, allowing for an optimized distribution of LEDs and light output per unit area. This may have the advantage of increasing the light density in a given treatment area, reducing the need for excessive numbers of light elements, thereby reducing excess heat generated in the system. Similarly, circular arrangement of LEDs in light emitter (130) may improve uniformity and coverage, particularly for treating circular or elliptical target areas. In some embodiments, light emitter (130) comprises LEDs with optical output power of approximately 1 W to approximately 10 W per LED element. In some embodiments, the total optical power output of light emitter (130) is in the range of approximately 10 W to approximately 100 W, depending on the treatment area size and application requirements.

As used herein, the term "light emitter" refers to any device or system capable of generating electromagnetic radiation within the spectrum suitable for PBM, which typically includes wavelengths in the visible range of approximately 400 nm to approximately 700 nm and near-infrared range of approximately 700 nm to approximately 1400 nm. For example, light emitter (130) may include LEDs at approximately 630 nm, approximately 660 nm, approximately 850 nm, approximately 940 nm, or any wavelength in between these values. LED arrays in light emitter (130) may include a variety of LEDs in terms of wavelength and other optical properties. For example, since penetration depth is a function of wavelength, with longer wavelengths generally penetrating deeper into tissue, it may be desirable to select LEDs of differing wavelengths within the same array in light emitter (130) to distribute the light energy at varying depths. Furthermore, light emitter (130) may be designed to operate with various power densities, for example in the range of approximately 1 mW/cm² to approximately 500 mW/cm², and energy dosages, for example in the range of approximately 1 J/cm² to approximately 100 J/cm², to tailor delivery of light for different tissue depths and application conditions as desired.

In some embodiments, light emitter (130) includes no lens. In some other embodiments, light emitter (130) may include at least one lens. A lens in light emitter (130) may refract and focus or disperse light rays as they pass through it. In some embodiments, light emitter (130) may include a collimator, diffuser, or optical interface to control beam divergence and ensure a spread of the light or uniform energy distribution across the target area. A diffuser in light emitter (130) may be an optical component that spreads out or scatters the light. A diffuser may help to even out intensity variations or irregularities in the light distribution, eliminating hot spots that could cause localized heating. In some embodiments, light emitter (130) includes optical fibers delivering light from a remote light source.

Transparent, as used herein, refers to a material property where light passes through the material without significant scattering or absorption, typically with transmittance greater than approximately 80% at the wavelengths of interest. Scattering may refer to the redirection of light as it interacts with particles, surfaces, or irregularities in a medium. Absorption may refer to the process in which a material takes in light or other electromagnetic energy, converting it into other forms of energy, such as heat. By using materials with low absorption at the relevant wavelengths for light emitter (130), absorption in medium (120) can be kept to a very low level, for example less than approximately 5% per centimeter of path length, providing a high transmission of the light to skin (110).

The operation of light emitter (130) may be performed by controller (150). Controller (150) may include various components for controlling light emitter (130), cooling components such as cooler (140), or user device (160). Controller (150) may include a processor, memory, storage, power circuitry, sensors, user interface components, or communication modules configured to operate light emitter (130) or cooler (140) or communicate with user device (160).

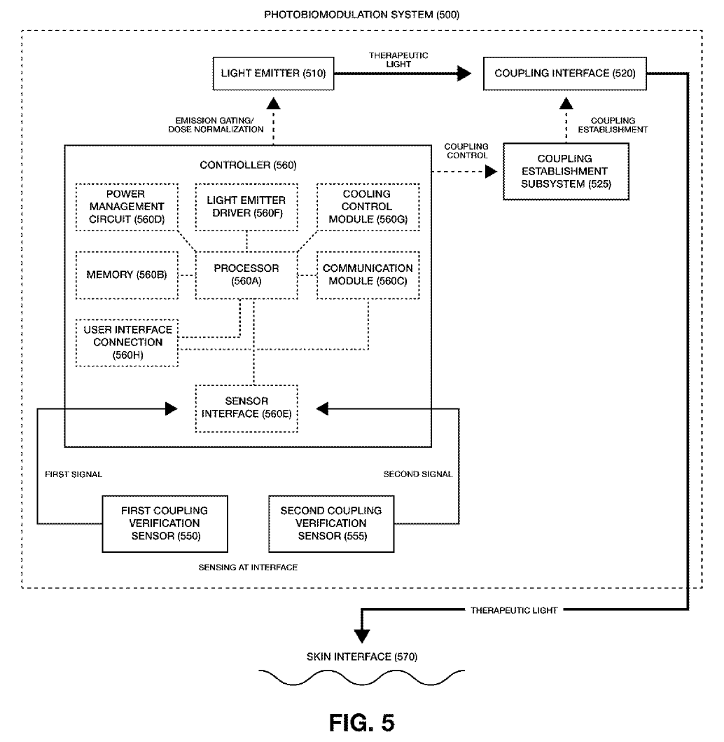

Referring now to FIG. 5, a processor in controller (560), which may correspond to processor (560A) in the controller (560), may include various types of processing devices suitable for controlling device operations. The controller (560) may be considered substantially the same controller (150) as shown in FIG. 1. Additionally, the light emitter (510) of FIG. 5 may be considered substantially the same light emitter (130) of FIG. 1. It will be apparent to those skilled in the art from this disclosure that FIG. 5 provides a diagrammatic view of the software components of FIG. 1. Therefore, the components of FIG. 5 may supplement the corresponding components of FIG. 1. The processor may include a microprocessor, microcontroller, digital signal processor, application-specific integrated circuit, field-programmable gate array, or any other processing device suitable for running control applications and implementing protocols. The processor may execute software instructions stored in memory (560B) to control light emitter (510) operation parameters, thermal management through cooling control module (560G), sensor data acquisition through sensor interface (560E), and communication functions through communication module (560C).

Memory (560B) in controller (560) may be a non-transitory memory, such as a flash memory, a random-access memory (RAM), electrically erasable programmable read-only memory (EEPROM), or other suitable memory types. The memory (560B) may be configured to store data, such as computer codes or instructions executable by the processor, protocols, sensor calibration data, user preferences, and history logs. Power management circuit (560D) may regulate power distribution to the various system components and manage battery charging if the device is battery powered.

Controller (560) may include communication modules, corresponding to communication module (560C), for communicating with user device such as the user device (160) of FIG. 1. Communication modules may be components that facilitate data exchange between controller (560) and external devices such as user device (160), monitoring systems, or networked platforms. In some embodiments, controller (560) may be configured to wirelessly communicate with user device (160). These modules may include wireless communication technologies, such as Bluetooth, Wi-Fi, near-field communication (NFC), cellular, or other wireless protocols to enable remote control, real-time monitoring, and synchronization with user devices or cloud services. In some embodiments, user device (160) is a mobile phone, tablet computer, laptop computer, smart watch or dedicated handheld controller. User device (160) may run an application programmed to communicate with controller (560) and provide a graphical user interface for device operation.

Referring again to FIG. 1, controller (150) may operate light emitter (130) to produce various temporal characteristics. For example, the light may be continuous wave (CW), pulsed, or modulated to optimize outcomes for specific applications. Continuous wave light may be light that is emitted continuously and uniformly over time, without interruption or pulsing. In some embodiments, controller (150) operates light emitter (130) in continuous mode. Pulsed light may be light emitted in short bursts or pulses, with periods of emission separated by intervals of no light emission. In some embodiments, controller (150) operates light emitter (130) in pulsed mode. The choice of pulse frequency, pulse duration, duty cycle, or other pulse parameters may be related to the desired effect from the light. In some embodiments, pulse frequency may range from approximately 1 Hz to approximately 10,000 Hz, and duty cycle may range from approximately 10% to approximately 90%.

Referring to FIG. 1, the controller (150) may operate cooler (140) through cooling control module (560G). Cooler (140) may be any cooling device or system used to remove excess heat from the system environment (100). For example, operation of light emitter (130) may generate excess heat as a byproduct of electrical-to-optical energy conversion. In some embodiments, cooler (140) may include one or more cooling fans as depicted in FIG. 1. Cooling fans may use ambient air flowing through the system to convectively remove heat from heat-generating components. In some embodiments, cooler (140) may include liquid cooling systems.

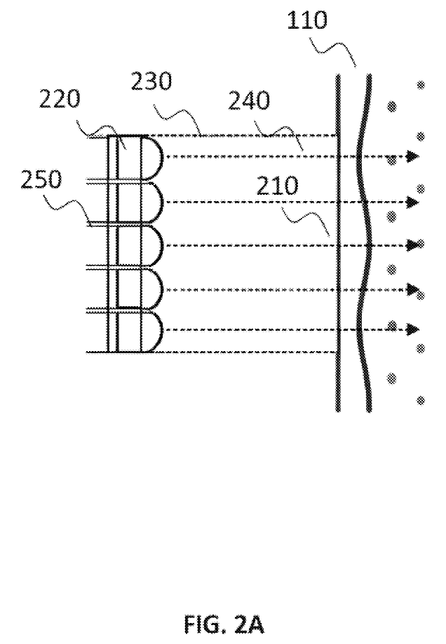

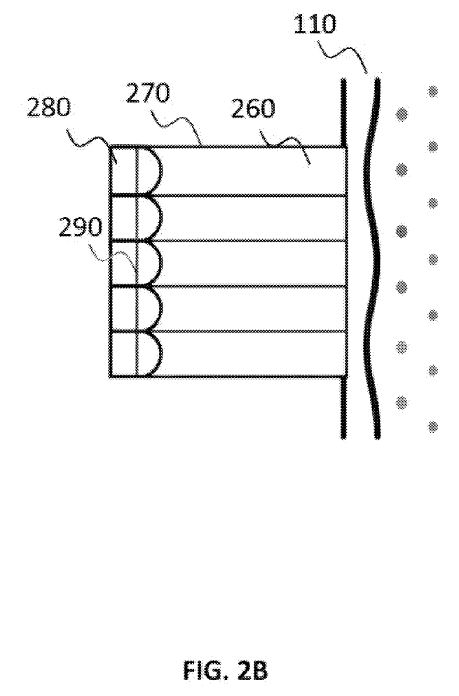

In some embodiments, microfluidic channels, corresponding to microfluidic channels (250) in FIG. 2A may be coupled to light emitter (130) or medium (120) for cooling. Microfluidic channels may be small, engineered pathways within a device that enable the controlled movement of fluids at small scales. These channels may have cross-sectional dimensions in the range of approximately 10 micrometers to approximately 1000 micrometers. The microfluidic channels may be pre-designed in medium (120) or positioned near the boundary of light emitter (130) or the interface with skin (110). The channels may circulate coolant to remove heat from light emitter (130) and transport the heated coolant to another location where it can be cooled before returning to the channels in a closed-loop cooling system. In some embodiments, the coolant may be water, glycol-water mixtures, or other suitable heat transfer fluids selected for thermal properties and compatibility with the device materials.

Referring to FIG. 1, in some embodiments, medium (120) is coupled directly to light emitter (130) on one side. On another side, medium (120) may contact skin (110). Coupling light emitter (130) to medium (120) may include mounting light emitter (130) directly to medium (120) by material bonding, adhesive attachment, mechanical fastening, or applying an encasing material surrounding part or all of both light emitter (130) and medium (120). In some embodiments, silicone may be used to surround light emitter (130) or medium (120). Silicone may be a durable, flexible, and biocompatible polymer material suitable for skin-contact device applications. In some embodiments, the coupling may be intended to be permanent to form an integrated device assembly. In some embodiments, medium (120) may be replaceable or interchangeable with different medium configurations. In some embodiments, the medium itself may be provided as a standalone replaceable component or consumable separate from the light emitter, allowing users to replace the medium while retaining the light emitter and other device components. Such replaceable medium configurations may be advantageous for hygiene, customization of optical properties for different applications, or cost-effective device maintenance.

In some embodiments, medium (120) includes silicone and hydrogel in combination. Hydrogel, as used herein, refers to a crosslinked hydrophilic polymer network capable of absorbing and retaining water while maintaining structural integrity. Hydrogel may be soft and pliable, allowing it to easily conform to irregular surfaces, such as the topology of skin (110) or the surface of light emitter (130). Hydrogel with medium to high viscosity, for example greater than approximately 1000 centipoise, may be chosen to reduce flow under compression during use. Hydrogel and silicone both have refractive indices in the range of approximately 1.35 to approximately 1.55, which is similar to that of water with a refractive index of approximately 1.33 or skin tissue with a refractive index of approximately 1.4 to approximately 1.55 depending on the tissue layer. By selecting materials for medium (120) with refractive indices closely matched to skin tissue, Fresnel reflection losses at the medium-skin interface may be reduced by a factor of 10 or more compared to an air-skin interface.

In some embodiments, hydrogel, corresponding to hydrogel (240) in FIG. 2A or hydrogel (260) in FIG. 2B, is in direct contact with skin (110). Hydrogel may help hydrate skin (110) during use, potentially enhancing light transmission through the superficial skin layers. The pliability of hydrogel allows for molding into the shape of the surface of skin (110), providing intimate contact over the entire area. This conformability is important to help remove air from the interface between medium (120) and skin (110), thereby increasing the efficiency of light transmission into skin (110). In some other embodiments, medium (120) includes a thin layer of silicone, corresponding to thin silicone layer (210) in FIG. 2A, configured to be in direct contact with skin (110). The thin silicone layer may have a thickness in the range of approximately 0.1 mm to approximately 2 mm. The silicone may encase hydrogel in the interior of medium (120), providing structural containment for the hydrogel. The thin silicone layer may provide support for the hydrogel and allow for easy cleaning after interfacing with skin (110), increasing the reusability and longevity of the device.

It should be understood that silicone and hydrogel are merely example materials for medium (120), and any highly conformable materials may be used in medium (120), provided they exhibit high optical transparency in the red to near-infrared (NIR) range of wavelengths and are biocompatible for skin contact applications. For example, polyurethane, polydimethylsiloxane (PDMS), polyethylene glycol, polyvinyl alcohol, or combinations thereof may be used in place of or in combination with hydrogel, silicone, or any other similar material in medium (120). The selection of materials may be based on factors including optical transmittance, refractive index, mechanical properties, biocompatibility, durability, manufacturing feasibility, and cost considerations.

In some embodiments, materials may be mixed into medium (120) to fine-tune refractive index for optimal index-matching. For example, water and glycerol may be used to control refractive index by varying their ratio, with higher glycerol content increasing refractive index. In some embodiments, biocompatible sugars such as sucrose or glucose may be used to adjust refractive index and viscosity. In some embodiments, biocompatible alcohols such as propylene glycol or ethanol may be used to modify refractive index. In some embodiments, medium (120) may include hydrophilic polymers such as polyvinylpyrrolidone (PVP), hyaluronic acid, or cellulose derivatives to modify viscosity and hydration characteristics.

In some embodiments, medium (120) may include a gradient or steps of refractive index changes over the length of medium (120), corresponding to gradient refractive index zones. For example, the refractive index may increase gradually from the interface with light emitter (130), where it may be approximately 1.4, to the interface with skin (110), where it may be approximately 1.5, providing a gradual optical transition that further reduces interface reflections. In some embodiments, gradient refractive index may be achieved by layered casting of materials with different refractive indices, diffusion-controlled polymerization, or incorporation of refractive index modifying additives at varying concentrations across the medium thickness.

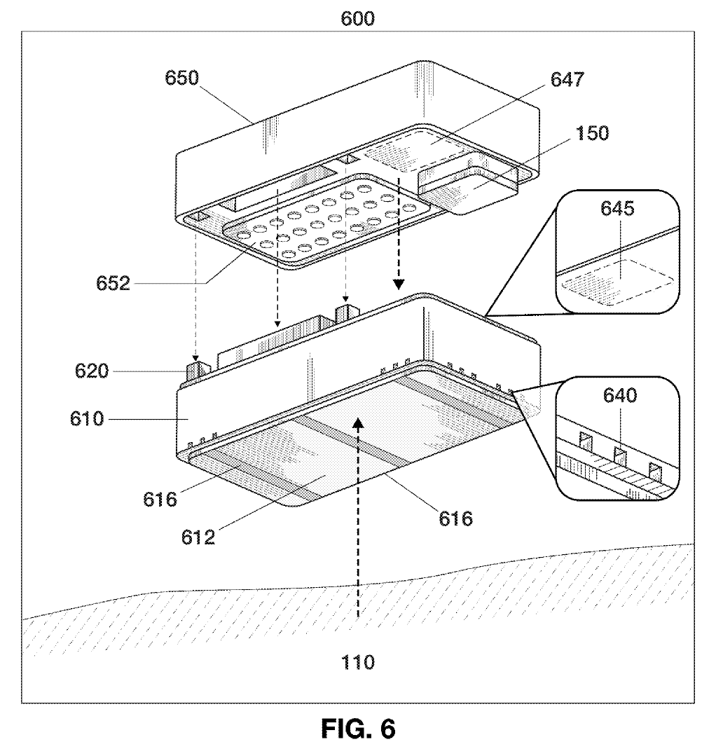

During use, compression and maneuvering of the device may deform medium (120) in undesirable ways, or cause irregularities in the delivery of light to skin (110). In some embodiments, medium (120) includes silicone baffles or structural support elements, corresponding to structural baffle element (616) in FIG. 6. Baffles may be structures that direct mechanical stresses and provide structural support within medium (120). For example, silicone baffles may direct mechanical stresses in medium (120) and prevent excessive deformation under compression. In some embodiments, silicone baffles or prongs, corresponding to silicone (290) forming baffles in FIG. 2B, may extend the length of medium (120) from light emitter (130) to skin (110) or to the thin silicone layer. These baffles or prongs may be columnar, tubular, prismatic, conical, or other suitable shapes, and may provide structural support to medium (120), particularly when the remainder of medium (120) is made of a softer hydrogel material. These baffles or prongs may also provide optical guiding properties. For example, if the baffle material has a higher refractive index than the surrounding hydrogel, the directionality of the baffles or prongs may act to guide light along the length of medium (120) through total internal reflection, which may reduce optical losses through the sides of medium (120). These baffles or prongs may also incorporate the microfluidic channels for cooling, providing multiple functions in a single structural element. The baffles or prongs may also create channels that allow for venting of air from the boundary between medium (120) and skin (110).

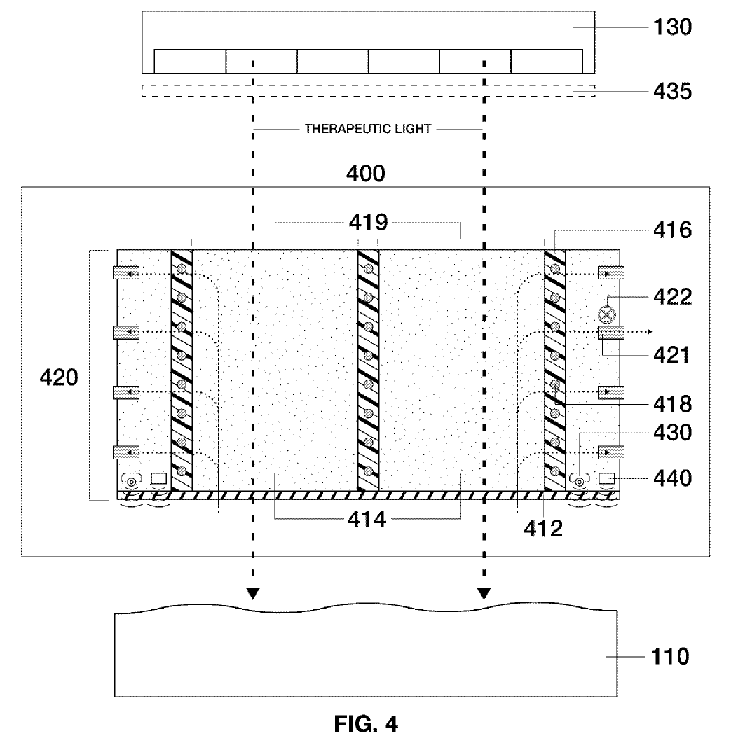

In some embodiments, a one-way valve, corresponding to one-way valve (422) in FIG. 4, is used to allow air to be removed from the interface between medium (120) and skin (110). It will be apparent to those skilled in the art from this disclosure that the coupling interface of FIG. 4 can be implemented with the system environment of FIGS. 1, 2A and 2B to deliver therapeutic light as desired. A one-way valve may be a valve that allows fluid, including air, to flow in one direction only. The one-way valve may enable the controlled evacuation of air trapped at the interface, which may otherwise create gaps, reduce contact uniformity, and interfere with the transmission of light through medium (120) to skin (110). By eliminating these air pockets, the valve helps to ensure a more consistent and effective optical coupling between medium (120) and skin (110), improving accuracy and efficacy of light delivery. The valve may include a membrane, flap, duck-bill structure, or ball-check mechanism that prevents air from re-entering the interface once it has been removed, maintaining a stable and sealed interface during operation. In some embodiments, the one-way valve may be connected to a peripheral ring chamber or air evacuation channel (408) around light emitter (130).

In some embodiments, air at the interface between medium (120) and skin (110) is evacuated manually, such as through a squeezable bulb, manual pump, or syringe mechanism. In some embodiments, air is evacuated using a vacuum system. For example, the vacuum system may be integrated into the device or may be a separate accessory. The vacuum may be operated by an electric motor or pump and may be automated under control of controller (150). Sensors, such as pressure sensors or contact sensors connected through sensor interface (510), may be used to detect when air has been substantially removed and a sealed condition has been achieved.

Medium (120) and light emitter (130) may be manufactured using various approaches. Two-shot molding or overmolding may mold silicone first then mold hydrogel into the silicone structure. Silicone coating may coat a thin layer of silicone onto pre-formed hydrogel using dip coating, spray coating, spin coating, or other coating techniques. Lamination would involve a thin silicone film laminated onto the hydrogel using an adhesive layer or heat bonding. Various polymerization reactions may be used to construct the hydrogel or silicone, and other materials may be used in combination with or in place of silicone and hydrogel. Photopolymerization using ultraviolet or visible light exposure may be used to cure certain hydrogel formulations. Thermal curing may be used for silicone materials.

Any ratio of silicone to hydrogel may be used in medium (120). By controlling the ratio of silicone to hydrogel, the mechanical and functional properties of medium (120) can be tailored for specific applications. In some embodiments, a ratio of the silicone to the hydrogel in medium (120) is between approximately 1/2 and approximately 3/4 by volume. For example, in a medium (120) having 100 mL total volume of silicone and hydrogel combined, silicone may constitute between approximately 50 mL and approximately 75 mL, with hydrogel constituting the remainder. This range of ratios may provide an optimal balance between structural support provided by silicone and conformability provided by hydrogel. In other embodiments, the silicone to hydrogel ratio may be outside this range depending on specific application requirements.

Referring now to FIG. 2A, an example of medium (230) with thin silicone layer (210) interfacing with skin (110) is illustrated, consistent with embodiments of the present disclosure. In this example, the light emitter is LED array (220). LED array (220) may emit light into medium (230) directly into hydrogel (240). After traversing hydrogel (240), light reaches thin silicone layer (210). At this point, air was removed so thin silicone layer (210) interfaces directly with skin (110) with low or substantially no residual air present, thereby increasing the efficiency of light penetrating into skin (110). LED array (220) may be cooled by microfluidic channels (250) positioned adjacent to or within the LED array structure.

Referring now to FIG. 2B, an example of hydrogel (260) in medium (270) interfacing directly with skin (110) is illustrated, consistent with embodiments of the present disclosure. In this example, the light emitter is LED array (280). Medium (270) may comprise silicone (290) encasing LED array (280) and forming baffles or prongs which traverse the length of medium (270) to the interface with skin (110). Hydrogel (260) fills the spaces between the silicone (290) baffles and may directly interface with skin (110) after removal of air at the boundary.

Referring now to FIG. 4, a detailed cross-sectional view of the coupling interface (420) is illustrated, consistent with embodiments of the present disclosure. The coupling interface (420) may include a light emitter (130) which can be configured as LED arrays as described above. The coupling interface (420) may comprise index-matching material layers that provide the optical coupling between the light emitter (130) and skin (110). Air evacuation channel (421) may allow air to be removed from the interface with skin. One-way valve (422) may prevent air from re-entering after evacuation. Structural baffle element (416) may provide mechanical support within the index-matching material. Microfluidic cooling channel (418) may circulate coolant for thermal management. The coupling interface can define gradient refractive index zones that provide gradual optical transitions between different refractive index regions.

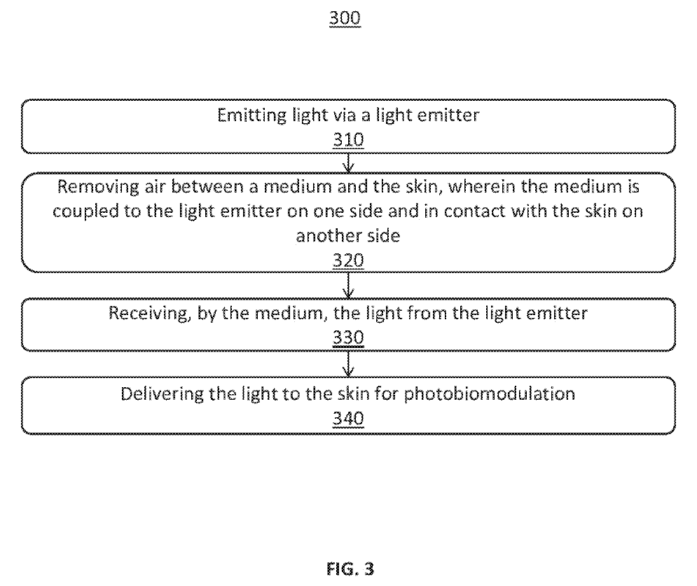

Referring now to FIG. 3, process (300) for delivering light to skin for PBM is illustrated, consistent with embodiments of the present disclosure. Process (300) may use any components or methods shown in FIGS. 1, 2A, 2B, 4-8, or described elsewhere in this disclosure.

In step (310) of process (300), a light emitter may emit light. The light emitter may be a laser, laser diode, LED, LED array, or any other source of light that emits in the red to NIR wavelength ranges suitable for PBM. The light emitter may be controlled by a controller, such as controller (150) in FIG. 1 or controller (560) in FIG. 5, or by user device (160), or may include built-in controls or preprogrammed functions.

In step (320) of process (300), air may be removed between a medium and skin, wherein the medium is coupled to the light emitter on one side and in contact with the skin on another side. The medium may include silicone, hydrogel, or any similar material with refractive indices in the range of approximately 1.35 to approximately 1.55, similar to that of skin or water. Air between the medium and skin may be removed manually through squeezing or pumping, or automatically through a vacuum system with sensors, to allow the medium to be in intimate direct contact with the skin surface. This air removal creates an index-matched optical pathway that reduces Fresnel reflection losses and other optical losses from the light emitter, thereby improving penetration of light deep into the target tissue.

In step (330) of process (300), light from the light emitter may be received by the medium. Light may traverse the substantially transparent medium without significantly altering its path or without noticeable absorption losses. Heat from the light emitter may warm the medium during operation, and removal of heat may occur by a cooler, such as cooler (140) in FIG. 1, through microfluidic channels, fans, or other thermal management systems.

In step (340) of process (300), light may be delivered to the skin for PBM. Once light has reached the skin-contacting side of the medium, it passes through the interface into the skin tissue. If air was removed, and the medium was index-matched to the skin with refractive indices within approximately ±0.1, then Fresnel reflection losses and other interface losses should be substantially reduced, thereby providing as much light as possible into the tissue for PBM.

The steps of process (300) may be performed in various orders or simultaneously. For example, air removal in step (320) may be performed before, during, or after light emission begins in step (310). The process may be repeated for multiple sessions, with the medium being cleaned between uses or replaced if disposable.

Referring now to FIG. 5, photobiomodulation system (500) is illustrated. System (500) comprises light emitter (510) configured to emit therapeutic light in wavelengths suitable for photobiomodulation are approximately 600 to 1200 nanometers. Coupling interface (520) is positioned to transmit therapeutic light from light emitter (510) to skin interface (170). Coupling establishment subsystem (525) is configured to establish enhanced optical coupling. Coupling verification sensors (550, 555) generate signals indicative of coupling quality. Controller (560) determines a coupling metric, gates therapeutic emission, and normalizes tissue-entry dose.

Light emitter (510) may comprise light-emitting diodes, laser diodes, or other suitable sources. In some embodiments, light emitter (510) comprises an array of LEDs arranged in geometric patterns optimized for uniform light distribution. Hexagonal arrangements may increase packing factor and optimize light output per unit area. Circular arrangements may improve coverage uniformity for round treatment areas. In some embodiments, the LED array comprises LEDs emitting at different wavelengths within the therapeutic range of approximately 600 to 1200 nanometers, such as 630, 660, 810, 850, or 1060 nanometers, to distribute therapeutic energy at varying tissue depths.

In some embodiments, light emitter (510) includes optical elements configured to modify beam characteristics before light enters coupling interface (520). A diffuser may spread light to achieve uniform illumination over the treatment area. A collimator may produce a more parallel beam to improve transmission efficiency through the coupling interface. A lens array may focus or defocus light from individual LEDs. In some embodiments, optical fibers deliver light from a remote source to the treatment interface. These optical distribution elements are optional system optimizations that work in conjunction with the verified coupling and dose normalization architecture.

Coupling establishment subsystem (525) may comprise any of: an evacuation subsystem including a seal, pump, and/or one-way valve configured to remove air from a boundary volume; a coupling medium delivery system configured to apply gel, fluid, or hydrogel; a mechanical compression system; or combinations thereof. The verified coupling state and dose normalization framework applies regardless of how enhanced coupling is established.

Coupling verification sensor (550) is configured to generate a signal indicative of optical coupling quality. In one embodiment, sensor (150) comprises an optical sensor detecting light reflected or backscattered from the interface during a low-power test emission. When coupling is poor, specular reflection is elevated; when coupling is good, specular reflection is reduced. Additional coupling verification sensor (555) may generate a second signal indicative of coupling quality based on a different physical property, such as pressure stability within a boundary volume, electrical impedance, or capacitance. By measuring different physical properties, the sensors provide independent verification evidence; degradation detectable by one sensor may not be detectable by the other, enabling comprehensive coupling assessment. In some embodiments, coupling verification is performed using a single modality (e.g., optical reflectance), while in other embodiments two or more modalities are used.

Controller (560) executes a coupling verification algorithm that determines a coupling metric from the sensor signals, compares the metric to a threshold, gates therapeutic emission, and controls emitter parameters for dose normalization. Controller (560) enforces safety limits: maximum emitted irradiance, maximum radiant exposure, maximum temperature, and maximum compensation factor. If compensation would exceed any limit, controller (160) aborts the session or prompts repositioning rather than operating unsafely. In some embodiments, the controller outputs a user prompt indicating repositioning, re-sealing, or re-application of coupling medium when coupling verification fails or when compensation would exceed safety limits. Therapeutic emission may also be inhibited based on fault conditions independent of coupling quality, including temperature limits, hardware faults, expired or incompatible cartridges, or user stop commands. In some embodiments, the controller records coupling metric values, coupling verification outcomes, test emission events, therapeutic emission events, and estimated tissue-entry dose values as a session log.

During treatment, controller (560) periodically re-verifies coupling state. If the coupling metric degrades below threshold, controller (560) pauses therapeutic emission, activates coupling establishment subsystem (525), re-verifies, and resumes. This closed-loop maintenance ensures consistent coupling throughout the treatment session.

Controller (560) permits test emission (low-power diagnostic pulses for coupling measurement) even while the therapeutic gate is closed. This enables continuous or periodic coupling assessment without requiring verified coupling as a prerequisite. Once test emission measurements confirm adequate coupling (metric meets threshold), the therapeutic gate opens and therapeutic emission proceeds. If coupling subsequently degrades, therapeutic emission pauses but test emission may continue for re-assessment.

The systems devices and method disclose treating optical coupling as a quantified operating state variable that is (i) objectively measured by at least one coupling verification sensor, (ii) used to gate therapeutic emission, (iii) used to normalize tissue-entry dose by controlling emitted parameters, and (iv) periodically re-verified and maintained during treatment with safety-bounded compensation. Hardware coupling structures, coupling media, evacuation mechanisms, and compression mechanisms are disclosed as example implementations for establishing coupling; the verified coupling state and dose normalization architecture applies regardless of which coupling establishment mechanism is employed.

Referring now to FIG. 4, system 400 including coupling interface (420) is illustrated in detail. Coupling interface (420) comprises an optically transmissive, deformable structure configured to reduce optical losses at the skin boundary relative to an air interface. The coupling interface (420) may comprise a deformable pad, a membrane, a rigid or semi-rigid optical window, a coupling medium layer, or combinations thereof. In some embodiments, coupling interface (420) comprises engineered structures that provide multiple functions: optical transmission, mechanical conformability, structural support, air removal pathways, and thermal management.

In some embodiments, coupling interface (420) comprises a silicone-hydrogel composite. Hydrogel region (414) comprises a hydrophilic polymer network capable of absorbing and retaining water while maintaining structural integrity. Hydrogel is soft and pliable, allowing it to conform to irregular skin surfaces and fill microtopographic features. Both hydrogel and silicone may have refractive indices between approximately 1.35 and 1.50, providing index matching to skin tissue. In some embodiments, thin silicone layer (412) is configured to be in direct contact with skin, encasing hydrogel region (414) in the interior. The thin silicone layer provides structural support for the hydrogel and allows easy cleaning for reusability. In other embodiments, hydrogel region (414) is configured to be in direct contact with skin, providing enhanced conformability and skin hydration during treatment.

In some embodiments, coupling interface (420) includes structural baffles (416). Baffles (416) comprise silicone or other suitable material extending through the thickness of coupling interface (420) from proximate light emitter (130) toward skin interface (110). Baffles (416) perform multiple functions. Mechanically, baffles (416) provide structural support to coupling interface (420), preventing collapse or excessive deformation under compression, particularly when surrounding material is softer hydrogel. Optically, baffles (416) may guide light along the thickness of coupling interface (420), reducing lateral spreading and light loss through the sides; the refractive index contrast between baffle material and surrounding hydrogel can provide waveguiding behavior and reduced lateral loss. For gas and/or fluid displacement, the spaces between baffles (416) define vent paths (419) that facilitate evacuation of air from the boundary between coupling interface (420) and skin (110), or that permit displacement and distribution of coupling medium when gel or fluid is employed. For thermal management, baffles (416) may incorporate microfluidic channels (418) for cooling fluid circulation.

In some embodiments, coupling interface (420) includes a gradient or stepped refractive index profile. The refractive index may increase gradually from the side adjacent to light emitter (130) toward the side interfacing with skin (110). This gradient configuration further reduces reflection losses by minimizing abrupt index changes.

Evacuation ring chamber (421) comprises a peripheral channel or cavity surrounding the treatment region. One-way valve (422) is fluidly coupled to ring chamber (421) and permits air egress while preventing air ingress. During device placement, air flows through vent paths (419) or channels between baffles (416), collects in ring chamber (421), and exits through one-way valve (422). In some embodiments, a collapsible reservoir is positioned near one-way valve (422); pressing the reservoir creates a partial vacuum that draws air from the interface. In some embodiments, a squeezable bulb or manual pump enables user-controlled evacuation. In some embodiments, a motor-driven pump provides automated evacuation controlled by controller (560).

Optical sensor element (430) is positioned to detect interface reflectance. Pressure sensor element (440) monitors pressure within the sealed boundary volume.

Optional diffuser (435) or collimator may be positioned between light emitter (130) and coupling interface (420) to shape the beam before it enters the coupling interface.

In some embodiments, enhanced coupling is achieved primarily through index-matching contact combined with air elimination at the skin interface. In this approach, the silicone-hydrogel coupling interface (420) provides a refractive index closely matching skin tissue, and the evacuation system (ring chamber (421), one-way valve (422), vent paths (419)) removes air from the boundary to establish intimate optical contact. The index-matching medium effectively replaces the air-skin interface with a medium-skin interface having reduced Fresnel reflection. This embodiment represents a preferred hardware implementation within the broader verified coupling and dose normalization framework. The coupling verification sensors (550, 555) confirm that air elimination has been successfully achieved and that the index-matching interface is functioning as intended; the controller (560) then uses the coupling metric derived from these measurements to gate therapeutic emission and normalize tissue-entry dose. Thus, the index-matching air-elimination hardware serves as the physical mechanism for establishing coupling, while the verification and normalization architecture ensures that coupling quality is objectively confirmed and dosimetrically compensated.

Coupling interface (420) may comprise silicone, hydrogel, silicone-hydrogel composites, polydimethylsiloxane, polyurethane, polyvinyl alcohol, or other optically transmissive biocompatible materials. In exemplary embodiments, coupling interface (420) is formulated to achieve a refractive index between approximately 1.38 and 1.45 and optical transmission greater than approximately 90 percent at therapeutic wavelengths within the range of approximately 600 to 1200 nanometers. Table 1 provides target specifications.

Target specifications for coupling interface (420) are as follows. Refractive index at 633 nanometers is between approximately 1.38 and 1.45, measurable by Abbe refractometer. Optical transmission at wavelengths from 600 to 1200 nanometers is greater than approximately 90 percent, measurable by spectrophotometer. Shore hardness is between approximately 15A and 35A, measurable by durometer. Biocompatibility is ISO 10993 compliant, verifiable by standard testing methods.

For skin-contact portions of coupling interface (420), refractive index and other optical properties may be adjusted using biocompatible additives. Suitable skin-contact additives may include glycerol, propylene glycol, polyethylene glycol of various molecular weights (e.g., PEG 200, PEG 400, PEG 600), sorbitol, maltitol, xylitol, mannitol, and other sugar alcohols; phosphate buffers, citrate buffers, and other biocompatible buffer salts for pH and ionic strength control. These additives are selected for established biocompatibility with prolonged skin contact and stability during treatment durations. The concentrations are selected to achieve target refractive index while maintaining material integrity and skin compatibility.

During manufacture of coupling interface (420), various processing aids and solvents may be employed to facilitate mixing, casting, curing, or coating operations. Such processing aids may include ethers, amides, ketones, esters, lactones, or other organic solvents suitable for polymer processing. These processing aids must be substantially removed from the finished coupling interface prior to skin contact use. Residual levels of processing aids should be verified to comply with ISO 10993 biocompatibility standards and applicable extractables/leachables testing requirements. The finished skin-contact product should contain only the biocompatible additives described above, with processing aids reduced to safe residual levels.

Coupling interface (420) may be manufactured using various processes. Two-shot molding or overmolding may first mold silicone structural components (including baffles) and then mold hydrogel material into the silicone structure. Pre-formed hydrogel components may be coated with thin silicone layers using dip coating, spray coating, or spin coating. Lamination may bond silicone film onto hydrogel using adhesives or thermal bonding. These manufacturing methods enable controlled formation of composite structures with defined silicone-to-hydrogel ratios, baffle geometries, and integrated channel networks.

The system 400 of FIG. 4 may include thermal management components. Microfluidic channels (418) integrated within baffles (416) or elsewhere in coupling interface (420) may circulate cooling fluid. Cooling fans may convectively remove heat from light emitter (130). Heat exchangers or liquid cooling systems may provide enhanced thermal management for high-power applications. Temperature sensors may provide closed-loop thermal control; controller (160) may reduce power if skin interface temperature exceeds a safety threshold.

The following describes one exemplary method for computing the coupling metric; other methods are possible and within the scope of the disclosure.

Controller (560) initiates a test emission at low power (non-therapeutic level) and measures reflectance signal R_meas from optical sensor element (430). If a reference region is available, controller (560) measures reference reflectance R_ref from the reference region. Normalized reflectance R_norm equals R_meas divided by R_ref (or divided by a stored baseline value if no reference region is present). The test emission may be performed while therapeutic emission is inhibited, and test emission energy is selected such that it does not materially contribute to the prescribed therapeutic tissue-entry dose. In some embodiments, the controller stores a pre-contact or initial baseline reflectance and uses the baseline as an alternative to the reference region measurement.

If pressure sensing is employed, controller (560) monitors pressure P over time after evacuation subsystem ceases active pumping. Pressure stability metric S_pressure is derived from the rate of pressure change; lower change rate indicates better seal integrity. S_pressure may be computed as one minus the ratio of measured leak rate to a maximum acceptable leak rate, bounded to the range zero to one.

The coupling metric M is computed by combining the normalized reflectance and pressure stability metrics. In one embodiment, M equals a weighted combination: M = w1 × (1 - R_norm) + w2 × S_pressure, where w1 and w2 are weights summing to one. Lower normalized reflectance (better optical coupling) and higher pressure stability (better seal) yield higher M. M is bounded to the range zero to one.

Controller (560) compares M to a threshold (e.g., 0.7). If M is below threshold, therapeutic emission is inhibited and the user is prompted to reposition or the system attempts additional evacuation. If M meets threshold, therapeutic emission is enabled.

For dose normalization, controller (160) determines a compensation factor F. In one embodiment, F equals M_nominal divided by M, where M_nominal is a target coupling metric (e.g., 0.9). If M is 0.8 and M_nominal is 0.9, then F equals 1.125, meaning at least one of emitted power, duty cycle, or exposure duration is increased by approximately 12.5% to compensate for reduced coupling. F is bounded by a safety ceiling (e.g., F_max = 1.5). If F would exceed F_max, controller (560) aborts and prompts repositioning rather than compensating into an unsafe regime.

Referring now to FIG. 6, replaceable cartridge system (600) is illustrated. Disposable coupling cartridge (610) comprises silicone-hydrogel pad (612) incorporating baffles (616), evacuation network (640), alignment interface (620), and optionally reference region (645). Reusable emitter head (650) contains LED array (652), controller (660), sensors, and optionally head-mounted reference region (647). Reference region (645) and/or reference region (647) have predetermined optical responses enabling calibration of coupling verification measurements.

Cartridge (610) may include an identification element storing calibration data, lot number, expiration date, and usage status. Emitter head (650) reads this data and may refuse operation with expired, previously used, or incompatible cartridges.

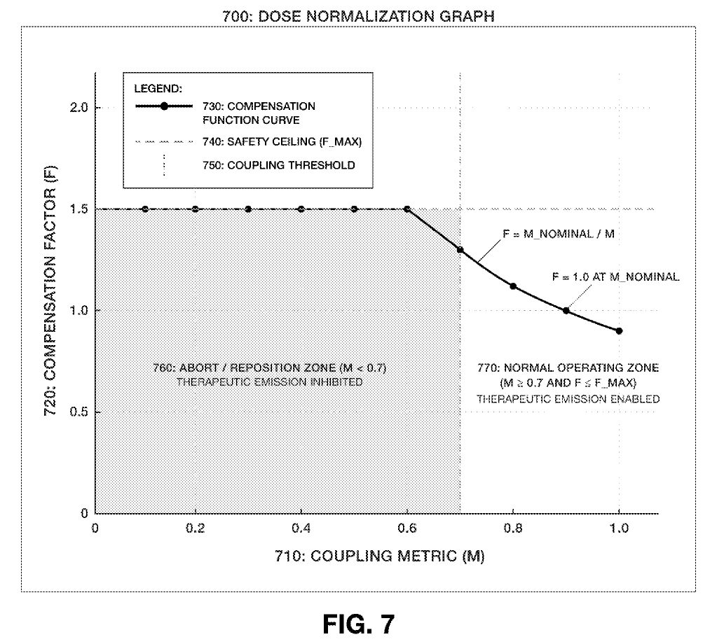

Referring now to FIG. 7, dose normalization graph (700) illustrates the relationship between the measured coupling metric and the controller compensation factor. Coupling metric axis (710) represents the coupling metric M with values ranging from zero to one, where higher values indicate better optical coupling. Compensation factor axis (720) represents the compensation factor F applied by controller (760) to normalize tissue-entry dose. Compensation function curve (730) depicts the inverse relationship F = M_nominal / M, where compensation increases as coupling quality decreases. Safety ceiling (740) represents the maximum permissible compensation factor F_max (e.g., 1.5), above which controller (760) aborts the session or prompts repositioning rather than compensating into an unsafe regime. Coupling threshold (750) represents the minimum acceptable coupling metric (e.g., 0.7) below which therapeutic emission is inhibited. Abort/reposition zone (760) represents the region where the coupling metric falls below threshold (750), indicating inadequate coupling that requires user intervention. Normal operating zone (770) represents the region where the coupling metric satisfies threshold (750) and compensation remains at or below safety ceiling (740), permitting therapeutic emission with dose normalization active.

Alternatively, controller (560) may use a lookup table mapping (R_norm, S_pressure) pairs to compensation factors, avoiding explicit formula computation.

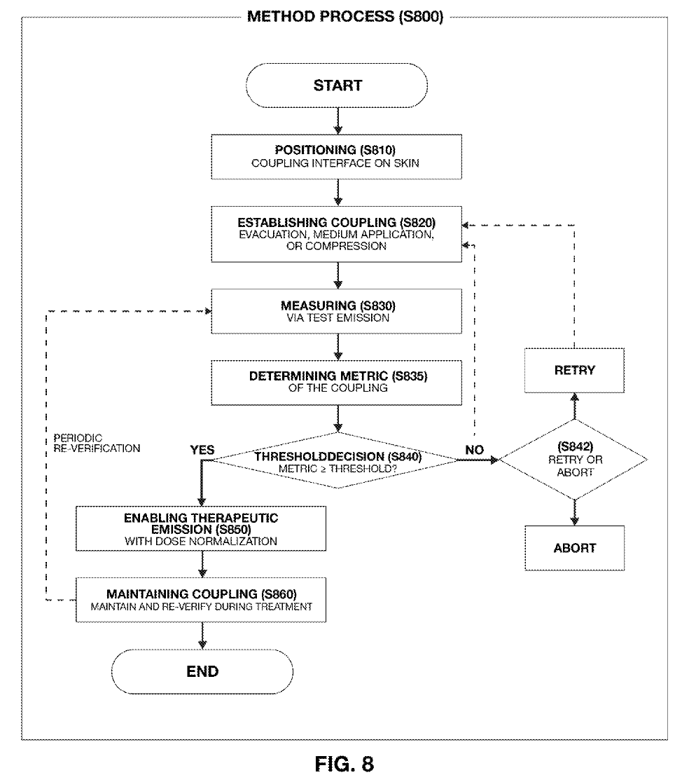

Referring now to FIG. 8, process (800) illustrates the method for photobiomodulation with verified coupling. In step (810), coupling interface is positioned on skin. In step (820), enhanced coupling is established (evacuation, medium application, compression, or combination). In step (830), coupling quality is measured via test emission (low-power, non-therapeutic). In step (835), coupling metric is determined. In decision step (840), controller verifies metric meets threshold. Safety check (842) verifies compensation within limits. If inadequate, the process loops to step (820) or aborts. In step (850), therapeutic emission is enabled with dose normalization. In step (860), coupling is maintained via periodic re-verification during treatment.

The foregoing description has been presented for purposes of illustration. It is not exhaustive and is not limited to the precise forms or embodiments disclosed. Modifications and adaptations will be apparent to those skilled in the art from consideration of the specification and practice of the disclosed embodiments.

Moreover, while illustrative embodiments have been described herein, the scope of any and all embodiments having equivalent elements, modifications, omissions, combinations of aspects across various embodiments, adaptations and alterations as would be appreciated by those skilled in the art based on the present disclosure.

The limitations in the claims are to be interpreted broadly based on the language employed in the claims and not limited to examples described in the present specification or during the prosecution of the application. The examples are to be construed as non-exclusive. Furthermore, the steps of the disclosed methods may be modified in any manner, including by reordering steps and inserting or deleting steps.

1. A system for delivering light to skin, comprising:
a light emitter configured to emit light; and
a medium coupled to the light emitter on one side and in contact with the skin on another side, air being removable from between the medium and the skin,
wherein the medium is configured to receive the light from the light emitter and deliver the light to the skin for photobiomodulation.

2. The system of claim 1, wherein the medium comprises silicone and hydrogel.

3. The system of claim 2, wherein a ratio of the silicone to the hydrogel in the medium is between approximately 1/2 and approximately 3/4 by volume.

4. The system of claim 2, wherein the medium comprises a thin layer of silicone configured to be in direct contact with the skin.

5. The system of claim 2, wherein the hydrogel is in direct contact with the skin.

6. The system of claim 1, wherein the light emitter is an array of light-emitting diodes.

7. The system of claim 6, wherein the array is arranged in a hexagonal shape.

8. The system of claim 6, wherein the array is arranged in a circular shape.

9. The system of claim 1, wherein the light emitter comprises at least one laser diode.

10. The system of claim 1, wherein the light emitter comprises at least one diffuser.

11. The system of claim 1, wherein the medium comprises silicone baffles.

12. The system of claim 1, further comprising microfluidic channels coupled to at least one of the light emitter and the medium for cooling.

13. The system of claim 1, further comprising a cooling fan.

14. The system of claim 1, further comprising a one-way valve to allow the air to be removed from an interface between the medium and the skin.

15. The system of claim 1, further comprising a controller configured to operate the light emitter and wirelessly receive control signals from a user device.

16. The system of claim 15, wherein the user device is a mobile phone, smart watch, hand controller, or tablet.

17. The system of claim 15, wherein the controller operates the light emitter in pulsed mode.

18. The system of claim 15, wherein the controller operates the light emitter in continuous mode.

19. A device for delivering light to skin, comprising:
a light emitter configured to emit light; and
a medium coupled to the light emitter on one side and in contact with the skin on another side, air being removable from between the medium and the skin,
wherein the medium is configured to receive the light from the light emitter and deliver the light to the skin for photobiomodulation.

20. A method for delivering light to skin for photobiomodulation, the method comprising:
emitting light via a light emitter;
removing air between a medium and the skin, wherein the medium is coupled to the light emitter on one side and in contact with the skin on another side;
receiving, by the medium, light from the light emitter; and
delivering the light to the skin for photobiomodulation.
